(19) United States
(12) Patent Application Publication
(10) Pub. No.: US 2011/0306917 A1
(43) Pub. Date: Dec. 15, 2011

(54) Activation of the alternative cellular energy (ACE) pathway in the therapy of diseases

(76) Inventor: William John Martin, South Pasadena, CA (US)

(21) Appl. No.: 12/802,605

(22) Filed: Jun. 9, 2010

(51) Int. Cl. A61N 5/06

(52) U.S. Cl. 604/20

(57) ABSTRACT

Alternative cellular energy pigments (ACE-pigments) provide a source of cellular energy other than that provided through the oxidative metabolism of foods, or in the case of plants and certain bacteria, through the process of photosynthesis. In some patients, ACE pigments exist in a form that can be further energized or activated using ultraviolet (UV) light, especially if the reaction is initially triggered by the presence of suitable dyes, such as neutral red. An alternative method has been described to activate the ACE pathway in humans and animals deprived of ACE by using natural or man-made sources of ACE products (enerceuticals), with or without the inclusion of a suitable dye, such as neutral red; and applying the material(s) to the skin, either directly or separated by an impermeable barrier; and illuminating the enerceutical with a UV light source. The process of activating the ACE pathway is evidenced by UV inducible fluorescence seen within areas of the patients' skin and/or mucus membranes. This fluorescence fades as the ACE pathway becomes fully activated. The present patent application shows that neutral red by itself in ethyl alcohol can be used as a suitable enerceutical. Activating the ACE pathway can have therapeutic benefits in various infectious and non-infectious diseases.

### Continuation of the Following Co-Pending Patent Applications:

Methods for Detection of Ultraviolet Light Reactive Alternative Cellular Energy Pigments (ACE-pigments) William John Martin Filed Dec. 24, 2007. Number 20090163831

Method of Assessing and of Activating the Alternative Cellular Energy (ACE) Pathway in the Therapy of Diseases. William John Martin Filed Jan. 17, 2008. Number 20090181467

Enerceutical activation of the alternative cellular energy (ACE) pathway in therapy of diseases. Filed Feb. 11, 2008 Number 20090202442

Enerceutical mediated activation of the Alternative Cellular Energy (ACE) pathway in the therapy of diseases. Filed May 8, 2008 Number 20090280193

Moringa Oil Mediated Activation of the Alternative Cellular Energy Pathway in the Therapy of Diseases Filed Feb. 8, 2010

### Previously Submitted but Now Abandoned Patent Applications

Ser. No. 10/044,683.Therapy of stealth virus associated cancers and other conditions using light. William John Martin. (Abandoned)

Ser. No. 10/047,313. Therapy of stealth virus associated cancers and other conditions using medium chain triglycerides. William John Martin. (Abandoned)

Ser. No. 10/050,232. Diagnosing and monitoring the therapy of stealth virus infections based on the detection of auto-fluorescent material in hair. William John Martin. (Abandoned)

Ser. No. 10/058,480. Therapy of stealth virus associated cancers and other conditions using magnetic energy. William John Martin. (Abandoned).

Ser. No. 10/164,258 Energy supportive therapy of stealth virus associated diseases. William John Martin. (Abandoned)

Ser. No. 10/174,466 Sound therapy of stealth virus associated diseases. William John Martin. (Abandoned)

Ser. No. 10/192,936 ACE-Pigments and humic acids as energy sources. William John Martin. (Abandoned)

Submitted: Ser. No. 10/______ Methods for Collection of Alternative Cellular Energy Pigments (ACE-pigments). William John Martin. (Abandoned)

Submitted: Ser. No. 10/______ Methods for Elimination of Toxic Alternative Cellular Energy Pigments (ACE-pigments) and for Their Replacement Using Activated Humates, including Humic and Fulvic Acids. William John Martin. (Abandoned)

### United States Patents (Awarded)

U.S. Pat. No. 5,985,546 Stealth virus detection in the chronic fatigue syndrome. William John Martin

U.S. Pat. No. 5,891,468 Stealth virus detection in the chronic fatigue syndrome. William John Martin

U.S. Pat. No. 5,753,488 Isolated stealth viruses and related vaccines. William John Martin

U.S. Pat. No. 5,703,221 Stealth virus nucleic acids and related methods. William John Martin

### PCT (Patent Cooperation Treaty)

WO 92/20797 Stealth virus detection in the chronic fatigue syndrome. William John Martin

WO 99/34019 Stealth virus nucleic acids and related methods. William John Martin

WO 99/60101 Stealth viruses and related vaccines. William John Martin

### REFERENCES TO PUBLISHED ARTICLES

### Alternative Cellular Energy Pigments (ACE-Pigments)

1 Martin W J. Alternative cellular energy pigments mistaken for parasitic skin infestations. Exp. Mol. Path 78: 212-214, 2005.

2 Martin W J. Alternative cellular energy pigments from bacteria of stealth virus infected individuals. Exp. Mol. Path 78: 217-217, 2005.

3 Martin W J. Progressive Medicine. Exp Mol Path 78: 218-220, 2005.

4 Martin W J, Stoneburner J. Symptomatic relief of herpetic skin lesions utilizing an energy based approach to healing. Exp. Mol. Path 78: 131-4, 2005.

5 Martin W J. Etheric Biology. Exp Mol Path 78: 221-227, 2005.

6 Martin W J. Stealth Virus Culture Pigments: A Potential Source of Cellular Energy. Exp. Mol. Pathol. 74: 210-223, 2003.

7 Martin W J. Complex intracellular inclusions in the brain of a child with a stealth virus encephalopathy. Exp. Mol. Pathol. 74: 179-209, 2003.

8 Martin W J. Photons and phonons: Theoretical aspects of biophysics and potential therapeutic applications. Proceeding of Neural Therapy Workshop on Sound and Light Therapy, Seattle, Wash., Feb. 21-23, 2003.

### Stealth Adapted Viruses

1 Martin W J Chronic fatigue syndrome among physicians. A potential result of occupational exposure to stealth viruses. Explore 2001; 10: 7-10.

2 Martin W J. Stealth Viruses. Explore 2001; 10: 17-19.

3 Dune G M, Collins R. Martin W J. Positive stealth virus cultures in multiple myeloma. A possible explanation for neuropsychiatric co-morbidity. Presented at the Am. Soc. Hematology annual meeting October 2000.

4 Martin W J. Chemokine receptor-related genetic sequences in an African green monkey simian cytomegalovirus-derived stealth virus. Exp Mol Pathol. 2000; 69:10-6.

5 Martin W J., Anderson D. Stealth virus epidemic in the Mohave Valley: severe vacuolating encephalopathy in a child presenting with a behavioral disorder. Exp Mol Pathol. 1999; 66:19-30.

6 Martin W J. Melanoma growth stimulatory activity (MGSA/GRO-alpha) chemokine genes incorporated into an African green monkey simian cytomegalovirus-derived stealth virus. Exp Mol Pathol. 1999; 66:15-8.

7 Martin W J. Bacteria-related sequences in a simian cytomegalovirus-derived stealth virus culture. Exp Mol Pathol. 1999; 66:8-14.

8 Martin W J. Stealth adaptation of an African green monkey simian cytomegalovirus. Exp Mol Pathol. 1999; 66:3-7.

9 Martin W J. Cellular sequences in stealth viruses. Pathobiology 1998; 66:53-8.

10 Martin W J. Detection of RNA sequences in cultures of a stealth virus isolated from the cerebrospinal fluid of a health care worker with chronic fatigue syndrome. Case report. Pathobiology. 1997; 65:57-60.

11 Martin W J., Anderson D. Stealth virus epidemic in the Mohave Valley. I. Initial report of virus isolation. Pathobiology. 1997; 65:51-6.

12 Martin W J. Simian cytomegalovirus-related stealth virus isolated from the cerebrospinal fluid of a patient with bipolar psychosis and acute encephalopathy. Pathobiology. 1996; 64:64-6.

13 Martin W J. Stealth viral encephalopathy: report of a fatal case complicated by cerebral vasculitis. Pathobiology. 1996; 64:59-63.

14 Martin W J. Genetic instability and fragmentation of a stealth viral genome. Pathobiology. 1996; 64:9-17.

15 Martin W J. Severe stealth virus encephalopathy following chronic-fatigue-syndrome-like illness: clinical and histopathological features. Pathobiology. 1996; 64:1-8.

16 Martin W J. Stealth virus isolated from an autistic child. J Autism Dev Disord. 1995; 25:223-4.

17 Gollard R P, Mayr A., Rice D A, Martin W J. Herpesvirus-related sequences in salivary gland tumors. J Exp Clin Cancer Res., 1996; 15: 1-4.

18 Martin W J., Glass R T. Acute encephalopathy induced in cats with a stealth virus isolated from a patient with chronic fatigue syndrome. Pathobiology. 1995; 63:115-8.

19 Martin W J, et al. African green monkey origin of the atypical cytopathic ‘stealth virus’ isolated from a patient with chronic fatigue syndrome. Clin Diag Virol 1995: 4: 93-103.

20 Martin W J. Stealth viruses as neuropathogens. CAP Today. 1994; 8: 67-70.

21 Martin W J. et al. Cytomegalovirus-related sequence in an atypical cytopathic virus repeatedly isolated from a patient with chronic fatigue syndrome. Am J Pathol. 1994; 145: 440-51.

22 Martin W J. Activation of the alternative cellular energy (ACE) pathway as natural therapy for patients with autism. Submitted Mar. 27, 2008 to J. Autism and Developmental Disorders.

23. Martin W J. Activation of the alternative cellular energy (ACE) pathway as natural therapy for herpes simplex and herpes zoster virus infections. Submitted Mar. 27, 2008 to J. Infectious Diseases and subsequently to J. Complimentary and Alternative Medicine (Not accepted).

24. Activation of the Alternative Cellular Energy (ACE) Pathway as Natural Therapy for Patients with Autism. Submitted originally to J. autism and Developmental Disorder, and in a revised and expanded form to Proceedings of the National Academy of Sciences and next to Autism. Not accepted by these journals.

### STATEMENT REGARDING FEDERALLY SPONSORED RESEARCH OR DEVELOPMENT

Not applicable: No Federal funding was received in support of this patent application.

### REFERENCE TO SEQUENCE LISTING, A TABLE OR A COMPUTER PROGRAM LISTING COMPACT DISK APPENDIX

Not applicable.

### BACKGROUND OF THE INVENTION:

The invention is based on the following broad conceptual understanding on how the body can acquire cellular energy other than through the oxidative metabolism of foods. Essentially, an alternative cellular energy (ACE) pathway has been identified that is mediated by the energy converting (transducing) properties of mineral containing complexes of organic molecules, arbitrarily termed alternative cellular energy pigments (ACE pigments). Cellular energy generated by this pathway can seemingly complement the chemical energy that is derived by living organisms from the metabolism of food. The ACE pathway is likely to contribute to various physiological functions of the body. Of particular relevance to this application, the ACE pathway is postulated to provide an auxiliary defense mechanism beyond that of the immunological system, such that an inadequacy of this pathway may limit the body's capacity to overcome various infectious diseases. The ACE pathway is also anticipated to be involved in the normal functioning of many organs, including the brain. Moreover, it is reasonable to presume that many illnesses, not necessarily of infectious origin, may place an added burden on the ACE pathway and that an inadequacy or deprivation of the ACE pathway may be a factor in delaying the normal disease recovery process. Conversely, augmenting or activation of an impaired ACE pathway may facilitate recovery from a wide range of various illnesses. Moreover, a fully functioning ACE pathway is likely to also be a factor in maintaining wellness, enhancing athletic performance, increasing cognitive abilities, etc.

The body's ACE pigments are envisioned as tiny batteries with differing levels of being “charged.” The working model postulates that uncharged or very poorly charged ACE pigments will not fluoresce when illuminated directly with an ultraviolet light, (for example a 13 watt Halco spiral UV light bulb.) They will, however, fluoresce when mixed with certain dyes, including neutral red (e.g. at 0.1 mg/ml; available from Dudley Chemicals, New Jersey). ACE pigments can also alter the fluorescence pattern of other dyes, including acridine orange and Stains-All. Once partially charged, ACE pigments will fluoresce directly when illuminated with UV light. The UV illumination is able to provide additional energy to the ACE pigments and when more fully charged, the ACE pigments will no longer fluoresce under UV illumination in either the presence or absence of neutral red dye. The three energy levels classification of ACE pigments has proven useful in the clinical assessment of patients. ACE pigments can be collected from areas of skin and skin lesions, hair, bodily fluids, including perspiration, saliva, urine and blood. In blood, the ACE pigments can be seen both within cells, especially in neutrophils and in serum/plasma. A simple home based fluorescence screening method is for people to collect material onto a gauze swab or Q-tip and to test fluorescence before and after the addition of neutral red dye. Direct fluorescence, indicating partially charged ACE pigments, can be tested for by directly illuminating the oral cavity, areas of skin and specific skin lesions of the test subject.

ACE pathway activation provides a therapeutic measure for use in patients with a wide range of illnesses. It can be achieved by direct UV illumination of ACE pigments in those patients in which their ACE pigments are partially charged. Even sunlight appears to be of benefit to such patients, possibly through the sun's ACE pigments activating capacity. For those patients with “uncharged” ACE pigments, that is, with ACE pigments that do not seemingly directly respond to UV light illumination, neutral red or other triggering dyes can be useful. Uncharged ACE pigments can accumulate in certain types of skin lesions and can also be present on normal appearing skin, possibly mainly derived from perspiration. Neutral red can, potentially, therefore, be applied to skin lesions or even to some areas of normal skin in patients with a deficiency of their ACE pathway. They can then be readily activated using UV light or even sun exposure. This reasoning explains some of the results reported in the 1970's that neutral red was effective in healing skin lesions caused by herpes simplex virus (HSV), when the area was illuminated with a white fluorescent light (which included a UV component). In collaboration with Dr. Jon Stoneburner, I was able to confirm the basic observations of UV inducible fluorescence and expedited healing of HSV and herpes zoster virus (HZV) skin lesions using UV light illumination of locally applied neutral red dye. As did earlier investigators, Dr. Stoneburner believed the neutral red dye was directly interacting with herpes viruses, leading to their photodynamic destruction. Based on my understanding of the ACE pathway, I independently proposed and confirmed that ACE pigments could still be used if first collected from a herpes skin lesion and placed in close proximity to, but not in direct contact with the herpes skin lesion. The ACE pigments were collected onto a surgical towel, which was placed over the skin lesion. The towel was stained with neutral red dye and illuminated with UV light. As anticipated, the portion of the towel on which material had been collected becomes fluorescent. Within minutes, as expected, the underlying skin lesion begins to show fluorescence when directly illuminated with the UV light. In most patients, careful observation will reveal discernable fluoresce in some of the surrounding skin areas, as can areas within the oral cavity (palate, tongue and cheeks). ACE pigments could also be collected from the genital areas of patients with a history of recurrent genital herpes infections. They could be used similarly to the pigments collected from active herpes skin lesions to provide systemic activation of the ACE pathway.

Interestingly, the cross activation of the body's ACE pathway is not mediated by the visible fluoresce of the collected dye-treated ACE pigments held against, but not in direct contact with the skin. This important observation was made by observing that effective cross activation of ACE pigments will still occur through a light impermeable barrier, such as black plastic sheeting or cotton toweling. Neutral red dye induced fluorescing saliva derived ACE pigments can be used on the toweling or on other material that is laid onto a skin lesion. This option provides an alternative to collecting materials from an active skin lesion or for treating in the absence of an active lesion. Similarly, ACE pigments can be collected from areas of normal appearing skin, especially where perspiration has collected.

Saliva, perspiration and even urine derived ACE pigments have been used externally, in conjunction with neutral red and/or acridine orange dyes plus UV illumination, in patients with other systemic illnesses, without direct skin lesions. Particular focus has been given to treating children with autism, a disease that I have attributed to a non-inflammatory brain infection (encephalopathy) caused by atypically structured (stealth adapted) viruses that are not effectively recognized by the cellular immune system. The success of this approach is seen not only in clinical improvements in behavior and cognition, but also in the induction of areas of skin fluorescence and, in particular, oral fluorescence.

As an alternative approach to using patient derived materials as the source of ACE pigments, I have sought materials and approaches that would provide systemic activation of the ACE pathway. I coined the term enerceuticals™ to describe non-human or animal derived materials that would either intrinsically possess ACE activity or be able to enhance the ACE pathway of a human or animal. Unlike pharmaceuticals that are primarily directed to the treatment of specific diseases, enerceuticals™ will potentially find benefit in the correction of all types of diseases in which there is a deficiency in the ACE pathway. Activating the ACE pathway can also potentially be useful to athletes aspiring to improve their performance. Enerceuticals™ are potentially active on plants and microorganisms, as well as humans and animals. Most importantly, enerceuticals™ do not need to physically localize to the site of a pathological process since they promote cellular healing by creating an energy field that extends to the pathological site.

I have evaluated various natural and formulated products for potential enerceutical™ activity. These have included humic and fulvic acids, terpene rich plant extracts, oil obtained from the leaves of moringa oleifera trees, organically complexed minerals and other substances. The basic screening test has been looking for microscopic effects observed in a droplet placed close by but not in direct contact with an activated enerceutical™ preparation. The test droplet can variously contain motile single cell microorganisms, small particulate materials, chemicals such as potassium permanganate that change color as a function of redox (reduction-oxidation potential) and other dyes. The test droplet can also be observed for the formation of microscopic bubbles (gas formation). These types of studies have been conducted for over 5 years. The studies have led to the concept that enerceuticals™ act as antennas, which can capture both conventional and possibly etheric energies, converting them to a form of energy that can be used by living cells. The cellular energy may work in part by increasing the availability of hydrogen atoms.

In seeking enerceutical™ materials, I was intrigued by my own finding that a potent therapeutic product called HANSI (homeopathic activator of the natural system immune), contained measurable quantities of xylocaine (Lidocaine). The original formulation of the product was said to have come from Argentina, possibly contributed to by a German immigrant. A form of HANSI has been produced for several years in the United States. The presence of xylocaine in HANSI was reminiscent of the therapeutic work of Ana Aslam on procaine, (a chemical closely related to xylocaine) and the field of Neural Therapy. I also observed that HANSI prepared for intramuscular and intravenous use would react vigorously when mixed with an alcoholic tincture of iodine giving a flurry of iodine containing droplets that would settle from the mixture. The droplets would interact even through they were not in direct contact with each other. The interactions included some droplets growing in size while nearby droplets would shrink and eventually disappear. Droplets would also occasionally align into an arcing pattern. The effect was ascribed to the xylocaine since some other preparations of HANSI intended for intranasal use did show the reaction and did not have detectable xylocaine. Furthermore, xylocaine, procaine, tetracaine and similar products all showed an intense interaction with iodine tincture. Iodine would also cross link neutral red causing particles to appear along with considerable formation of scattered microscopic gas bubbles. Iodine has also been shown to interact with several herbal preparations, including several of those said to be in homeopathic amounts in HANSI (since called Enercel) and a terpene rich plant extract called EH-101.

The xylocaine—tincture of iodine droplets were settling to the bottom of the containers within a water phase with an overlying layer of alcohol. The droplets could be dispensed if the water was removed and alcohol reapplied. A similar separation of water from alcohol could be demonstrated by dissolving xylocaine in alcohol (at approximately 20 mg/ml or higher). By progressively adding water, the xylocaine would form a flocculent and/or needle shaped crystals, if done slowly. The alcohol would separate from this material rising to the surface of the solution.

The next line of inquiry was to test various xylocaine containing herbal formulations for their capacity to yield UV fluorescing materials when mixed with neutral red dye. The xylocaine was used at 0.5 to 2% with some loss due to flocculation during the preparation. The initial selection of herbal products was based on the components said to be included in HANSI and a related product from Brazil called Canova. They included approximately equal amounts of 3×(3 times 10 fold) dilutions in 10% alcohol of Aconite napellus, Byronia alba, Chelidonium major, Cinamium, Pulsatilla, Lachesis mutus, Thuja occident, Armica Montana and Arsebucan album (Obtainable from Boiron, Newtown Square, Pa., as homeopathic tinctures). Varying amounts of differing mineral were added to the solutions, including potassium chloride, calcium chloride, magnesium sulfate, phosphorous, silica and sodium arsenate. The preliminary criteria of judging the possible therapeutic potency of the solutions included i) bright UV fluorescence when neutral red was added. ii) A vigorous flurry of activity with prominent formation of xylocaine needles as the alcohol would evaporate from the solution. iii) Formation of gas bubbles in the fluid. The criteria were then extended to: i) The capacity to either induce gas bubble formation, actual movement of suspended particles or aberrant motility of microorganisms swimming in the water droplet placed close by, but not in direct contact with, the UV illuminated fluorescing solution. A solution with these properties was prepared in early 2008 and made available for clinical testing. It proved very effective in treating patients with active herpes skin lesions and also in treating patients without skin lesions. The patients included those with a history of recurrent herpes simplex infections, patients with active shingles and those with post herpetic neuralgia, patients with emphysema and a series of patients with either presumed or known stealth adapted virus infections. Included was a patient with chronic fatigue syndrome, several patients with Morgellon's disease (whose ACE pigments are typically misidentified as parasites), over 10 children with autism, a patient with emphysema and several women with cellulitis. All treated patients showed marked clinical benefits from a single, or in the case of autistic children, repeated treatments. Moreover, areas of skin and oral cavity UV inducible fluorescence were observable in the patients who were examined for this phenomenon during treatment.

In extending the initial study, I learned from parents of autistic children that the solution had lost its therapeutic activity. Confirmation was obtained from parents who had earlier experienced considerable improvements using the same batch of solution. The expired solution would still fluoresce with neutral red but indirect effects on other droplets could not be shown.

Neutral red fluoresces brightly when dissolved in ethanol. Unbeknown to me at the time when I made this observation, it had been briefly reported years earlier. Neutral red fluorescence also occurred when it is dissolved in methanol, propanol and even acetone. I originally dismissed the importance of this observation until I noted that a fluorescing ethanol solution was clearly affecting the motility and survival of paramecia (a unicellular microorganism) in an adjacent dish. This observation led to the following studies.

### BRIEF SUMMARY OF THE INVENTION

Neutral red dissolved in ethanol will fluoresce when illuminated with ultraviolet (UV) light. If the reaction is allowed to occur within a plastic container laid onto the skin surface of a patient with a deficient alternative cellular energy (ACE) pathway, it can lead to activation of the patients ACE pathway with resulting therapeutic benefits. Essentially, neutral red was potentially acting as an enerceutical™ product when it is dissolved in ethyl alcohol. Note that this invention is a continuation of research on stealth adapted viruses and on the alternative cellular energy (ACE) pathway that has been ongoing for more than 20 years. Additional information on the research and additional supporting information on the present patent application is provided in the cited pending and abandoned patent applications and in the cited references. These materials are incorporated into the present application by reference.

### BRIEF DESCRIPTION OF THE DRAWINGS

Not Applicable and none included

### DETAILED DESCRIPTION OF THE INVENTION

Rather than relying upon neutral red to interact directly with ACE pigments derived from a patient, I have discovered that UV light illuminated neutral red dissolved in ethyl alcohol can be used to help activate the body's own ACE pigments. In a preferred embodiment, approximately 2-5 mg of neutral red freshly dissolved in 10 ml of ethyl alcohol is placed in a re-sealable plastic bag with or without a small sheet of absorbent paper to help evenly distribute the fluid. The plastic bag is placed onto the surface of the body to ensure direct contact, using tape if necessary. The bag is illuminated with a 13 watt spiral ultraviolet light bulb positioned close to the solution which will show bright fluorescence in a darkened room. The therapy session will typically last from 30-60 minutes. The effectiveness of the procedure can be monitored by occasionally observing the inside of the patient's mouth and other areas of the patient's skin for UV inducible fluorescence. If fluorescence is not present prior to beginning the treatment session and if the person has a deficiency in the ACE pathway, it is expected that oral fluoresce will develop by the end of the treatment session. Indeed, the development of oral or discernable skin fluorescence in an individual undergoing the procedure provides a further indication that the individual has a deficiency in the ACE pathway, which can be potentially corrected using the procedure. In some patients, the induced oral fluorescence persists for several hours and even beyond a day. For patients in whom some fluorescence is present prior to starting the treatment, for example residual fluorescence from an earlier treatment, the intensity of the fluorescence would be expected to increase with the additional therapy. Patches of developing skin fluorescence can also be observed during therapy, especially in areas beneath and adjacent to those being treated. Preferred sites of treatment in patients without a localized skin lesion are the soles of the feet, palms of the hand, face (using UV light blocking goggles, with or without the mouth being held open during the procedure) and nape of the neck. The soles, palms and face are chosen since they are richly innervated and there are data that ACE pigments may be transported through nerves.

Experiments were conducted to determine if adding the patient's spit to the ethanol solution of neutral red might enhance its effectiveness. This did not appear to offer any additional benefit, either in terms of improved behavior or cognitive performance or inducible oral or skin fluorescence.

Experiments were also conducted to help clarify the possible mechanism by which the xylocaine-herbal formulations may have been operating. The fluorescence of neutral red in varying concentrations of alcohol in water was compared using differing sources of water and water solutions containing varying herbal products, which are typically dissolved in an alcohol containing solution. The picture is emerging that the alcohol mediated fluorescence capacity is retained much more effectively in some herbal formulations than in others. Possibly by binding to the water, some of the herbal materials can loosen the water—alcohol bonding, leading to more available domains or clusters of reactive alcohol. The situation is complicated by the effect of pH on neutral red fluorescence and the apparent presence of ACE-like pigment materials in some municipal water supplies.

As a confirming method of analysis, plastic dishes containing neutral red in either 100% ethyl alcohol or lower concentrations of alcohol—herbal formulations are laid under or over another dish containing motile single cell microorganisms. Varying effects can be seen on the patterns of motility of the microorganisms. Initially, there appears to be heightened and erratic movements, later the distribution of the microorganisms within the dish becomes uneven with major areas of high concentration. Eventually, there is a loss of movement and actual death. These changes are not seen with control dishes. Another observed change with neutral red dye (and also with some humic acid preparations) dissolved in alcohol solutions is the formation of gas bubbles, likely to be hydrogen. Similar observations have been seen with patient derived ACE pigments.

With better instrumentation, it is very likely that actual biophysical properties of enerceuticals,™ including neutral red in ethyl alcohol, can be better characterized and their therapeutic performance greatly enhanced. Methods are underway to test alternative alcohol or other solvent and to include small quantities of various herbal products into the alcohol containing solution to which neutral red and/or possibly additional dyes can be added. At the present time, however, it is adequate to use only an ethyl alcohol solution of neutral red to observe activation of the ACE pathway and a beneficial clinical effect.

The principles, preferred embodiments and modes of operation of the present invention have been described in the foregoing specification. The invention, which is intended to be protected herein, is not to be construed as limited to the particular ethyl alcohol neutral red dye solution disclosed. It is to be regarded as illustrative of all solutions and/or chemical materials that can be used to activate the body's ACE pathway. Nor is autism to be considered the only type of illness to be treated using the protocol. Indeed, it has already found successful application in a patient with a bipolar psychosis and in another patient with the chronic fatigue syndrome. The method is immediately applicable to the therapy of herpes infections (herpes simplex virus, herpes zoster virus, cytomegalovirus, Epstein Barr virus and human herpes virus-6). Indeed, the method is applicable in all patients presumed or shown to have a deficiency in the ACE pathway. The basic claim is, therefore, for the broader issue of achieving therapeutic benefit through activation of the ACE pathway, with an example being the use of an alcoholic

As will be described in a separate patent application, alcohol induced neutral red fluorescence, can also be used by patients in the monitoring of their own ACE pathway. In other words, dipping a gauze pad or Q-tip into alcohol can provide a convenient positive control to show neutral red fluorescence. Additional advantages and modifications of the basic tenets disclosed in the present patent application will readily occur to those skilled in the art and especially upon practicing the currently described methods. Many variations and changes may be made without departing from the scope and spirit of the invention as encompassed by the appended claims.

What I claim as my invention is:

1. A method for activating the alternative cellular energy (ACE) pathway in an ACE energy deprived human or animal subject comprising the application of ACE generating and/or stimulating products, termed enerceuticals, directly to the skin or to a fabric or other material or container that is placed onto the skin, without or with the addition of a suitable dye solution that can activate ultraviolet (UV) light inducible fluorescence of the enerceutical; such that UV illumination of the enerceutical or enerceutical-dye mixture will evoke UV inducible fluorescence in areas of the skin and/or within the mouth of the subject with the goal of assisting in the healing of a disease process affecting the subject.

2. A method of accessing the status of the alternative cellular energy (ACE) pathway in a human or animal subject by employing the method of claim 1 and determining whether this method induces the appearance of UV inducible fluorescence within the skin and/or mouth of the subject, such that the appearance of skin or palatal fluorescence is indicative of ACE energy deprivation in the subject.

3. The method of claim 1 in which the enerceutical is an alcohol containing solution of neutral red dye, which is able to fluoresces when illuminated with ultraviolet light.

4. The method of claim 3 in the concentration of alcohol used is sufficient to cause the fluorescence of dissolved neutral red dye under UV light illumination.

5. The method of claim 3 in which a lower concentration of alcohol is used than specified in claim 4, but one in which the addition of herbal or other products, used alone or in varying combinations, restores the fluorescence of any dissolved neutral red dye under UV light illumination.

6. The method of claim 3 in which the additional herbal and other products added to the alcohol solution are selected from among the following compounds; xylocaine, Aconite napellus, Byronia alba, Chelidonium, major Cinamium, Pulsatilla, Lachesis mutus, Thuja occident, Armica Montana, Arsebucan album, potassium chloride, calcium chloride, magnesium sulfate, phosphorous, silica and sodium arsenate.

7. The method of claim 3 in which the neutral red is used at a concentration ranging from 0.05 to 5.0 mg/ml.

8. The method of claim 3 in which the alcohol is ethyl alcohol.

9. The method of claim 1 in which the diseases is occurring in a patient with a deficiency in his or her alternative cellular energy (ACE) pathway, as shown by direct and/or neutral red dye inducible fluorescence of any material, including bodily fluids, obtained from the patient.
